(19) United States
(12) Patent Application Publication
(10) Pub. No.: US 2022/0291217 A1
(43) Pub. Date: Sep. 15, 2022

(54) GASTRIC CANCER MARKER AND EXAMINATION METHOD USING SAME

(71) Applicants: JAPANESE FOUNDATION FOR CANCER RESEARCH, Tokyo (JP); TOSOH CORPORATION, Shunan-shi, Yamaguchi (JP)

(72) Inventors: Koji Ueda, Tokyo (JP); Naomi Ohnishi, Tokyo (JP)

(21) Appl. No.: 17/639,979

(22) PCT Filed: Sep. 4, 2020
(86) PCT No.: PCT/JP2020/033551

§ 371 Date: Mar. 3, 2022

(30) Foreign Application Priority Data
Sep. 5, 2019 (JP) 2019-161687

(51) Int. Cl. G01N 33/574

(52) U.S. Cl. CPC G01N 33/574

(57) ABSTRACT

Exosomes were purified from the sera of patients with gastric cancer and healthy subjects by using size-exclusion chromatography, and novel markers were obtained through mass spectrometry. In the patients with gastric cancer, 40 proteins with enhanced expression and 4 proteins with decreased expression can be suitable markers for detecting gastric cancer. Particularly, the detailed analysis of CA1, including its function, showed that gastric cancer could be detected with high sensitivity.

### TECHNICAL FIELD

The present invention relates to a gastric cancer marker contained in an exosome and an examination method using the same.

### BACKGROUND ART

In Japan, the probability of developing cancer in one's lifetime is said to be one in two due in part to the aging of the population. In particular, the number of patients suffering from gastric cancer remains high, and in terms of the estimated number of cancer incidence cases by site, the stomach is the first site for men with 87,800 cases and the third site for women with 40,900 cases following breast and colon/rectum (Foundation for Promotion of Cancer Research (FPCR), CANCER STATISTICS IN JAPAN 2018).

Although the number of deaths from gastric cancer in Japan has been decreasing every year as a result of advances in early detection and treatment of gastric cancer through screening, many patients need to be monitored for recurrence, metastasis, and the like because of the large number of patients suffering from the disease. In cases such as recurrence, where primary cancer has already been removed, it is not usual to collect diseased site and examine it again. In order to detect metastasis at an early stage, it is considered effective to periodically examine for biomarkers present in body fluids such as blood.

Currently, carcinoembryonic antigen (CEA) contained in serum is used as a biomarker. CEA is a representative tumor marker with enhanced expression in various cancers and is not a marker specific to gastric cancer. In addition, its expression is not enhanced in all patients with tumors, as there are large individual differences.

Extracellular vesicles, especially exosomes, have been intensively studied in recent years and their functions have been elucidated. Exosomes are lipid bilayer membrane vesicles of 40-100 nm in size and stably present in body fluids such as blood and urine. Exosomes are secreted from most cells, and proteins, miRNAs, and mRNAs encapsulated therein are said to reflect properties of the cells from which they are derived. Thus, exosomes secreted from diseased cells such as cancer contain disease-specific markers. Therefore, exosome analysis is useful for the diagnosis of diseases, especially cancer.

It is known that exosomes secreted from cancer cells not only encapsulate molecules involved in oncogenesis, but also mediate cancer invasion, metastasis, immunosuppression, and angiogenesis. In other words, exosomes also function as communication tools between the cells that secreted them and the cells that ingested them.

In addition, as described above, exosomes can be prepared in a minimally invasive and noninvasive manner to perform diagnosis since they are contained in body fluids such as blood and urine. This is a great advantage to the patients since it can be an alternative to tissue biopsy in cases where periodic testing is required after surgery or where it is difficult to collect a diseased site. Exosomes are also a potentially useful resource for early cancer diagnosis since cancer cells are considered to secrete distinctive exosomes, even in early-stage cancers. Thus, the use of exosomes in body fluids as biomarkers for diseases such as cancer has been considered (Patent Literatures 1 and 2).

### CITATION LIST

### Patent Literature

Patent Literature 1: Japanese Translation of PCT International Application Publication No. 2016-520803
Patent Literature 2: Japanese Translation of PCT International Application Publication No. 2017-526916

### SUMMARY OF INVENTION

### Technical Problem

However, there is a problem of inclusion of serum proteins and the like when separating exosomes from body fluids containing large amounts of proteins, such as serum proteins, and analyzing them. Since there is a tiny amount of exosomes in body fluids as well as a very small amount of proteins encapsulated therein, the inclusion of serum protein makes it difficult to detect the proteins encapsulated in the exosomes. In addition, since almost all cells secrete exosomes, the amount of exosomes secreted from normal cells, which are overwhelmingly more abundant than diseased cells, is considered to be larger. Therefore, exosomes have not yet been used as markers in actual clinical practice due to the need to improve the accuracy of detection.

An object of the present invention is to provide a novel marker for gastric cancer, for which no suitable marker has been available. Another object is to examine for gastric cancer using this marker. Besides, the present invention relates to a method for easily and reproducibly purifying an exosome from a body fluid such as serum and using the purified exosome to search the marker.

### Solution to Problem

The present invention relates to a marker for detecting gastric cancer, an examination method, and a method for searching a novel marker from exosomes in blood.

(1) An examination method for gastric cancer, comprising examining for expression of at least one protein listed in Table 1.
(2) The examination method for gastric cancer according to (1), wherein the protein expression is to detect an amount of protein encapsulated in an exosome in a blood sample.
(3) The examination method for gastric cancer according to (2), wherein the blood sample is serum or plasma.
(4) The examination method for gastric cancer according to any one of (1) to (3), wherein detection of the protein is performed by mass spectrometry.
(5) The examination method for gastric cancer according to any one of (1) to (3), wherein detection of the protein is performed using an antibody.
(6) The examination method for gastric cancer according to (1), wherein detection of the protein is performed by tissue staining.
(7) The examination method for gastric cancer according to any one of (1) to (6), wherein the protein is carbonic anhydrase-1 (CA1).
(8) A search method for a disease marker, comprising: isolating exosomes from blood samples of patients suffering from a certain disease and of healthy subjects each by size-exclusion chromatography and identifying proteins with differences in expression between the patients and the healthy subjects by mass spectrometry to search a novel diseases marker.
(9) A biomarker for detecting gastric cancer listed in Table 1.
(10) The biomarker according to (9), which is contained in an exosome.
(11) The biomarker according to (10), wherein the exosome is a sample derived from blood.
(12) The biomarker according to (11), wherein the sample derived from blood is serum or plasma.
(13) The biomarker according to any one of (10) to (12), wherein the exosome is purified by size-exclusion chromatography.
(14) The biomarker according to any one of (9) to (13), wherein the biomarker is CA1.
(15) The biomarker according to (14), wherein the biomarker is indicated to be involved in apoptosis or anoikis resistance.
(16) A method for diagnosing gastric cancer, comprising detecting gastric cancer by collecting blood from a subject, detecting at least one biomarker listed in Table 1, and quantifying an amount of the biomarker.
(17) The method for diagnosing gastric cancer according to (16), wherein the biomarker is CA1.
(18) The method for diagnosing gastric cancer according to (16) or (17), wherein the detection of the biomarker is by mass spectrometry or immunological detection method using an antibody.

### BRIEF DESCRIPTION OF DRAWINGS

FIG. 1a shows that exosomes are purified by size-exclusion chromatography and is a diagram illustrating results of ELISA analysis.

FIG. 1b shows that exosomes are purified by size-exclusion chromatography and is a diagram illustrating results of the Western blotting analysis.

FIG. 2a is a diagram illustrating a volcano plot of exosomal proteins in which differences were found between patients with gastric cancer and healthy subjects. CA1 with the biggest difference in expression between the patients with gastric cancer and the healthy subjects is indicated by an arrow.

FIG. 2b is a diagram of 44 proteins with differences in expression levels between patients with gastric cancer and healthy subjects, analyzed by partial least squares regression.

FIG. 2c is a diagram illustrating the absolute quantification of exosomes in healthy subjects and patients.

FIG. 2d is a diagram of CA1 levels in serum exosomes compared between healthy subjects and patients with gastric cancer.

FIG. 2e is a diagram illustrating results of the quantification of CA1 levels in healthy subjects and patients with gastric cancer at each stage.

FIG. 2f is a diagram illustrating ROC curves indicating the sensitivity and specificity of CA1.

FIG. 2g is a diagram illustrating results obtained by purifying exosomes from patients with gastric cancer and healthy subjects by size-exclusion chromatography and analyzing CA1 in each fraction by Western blotting.

FIG. 3a is a diagram of CA1 expression analyzed in tissues.

FIG. 3b is a diagram illustrating staining intensities scored by anti-CA1 antibody in normal mucosal tissue, adenocarcinoma, undifferentiated carcinoma, and signet ring cell carcinoma.

FIG. 4a is a diagram of analysis of CA1 expression in gastric cancer cell lines.

FIG. 4b is a diagram of analysis of resistance to induction of apoptosis by forcing CA1 expression in SNU-1 cells that have not expressed CA1.

FIG. 4c is a diagram of analysis of resistance to induction of apoptosis by adding exosomes encapsulating CA1 to a culture medium of MKN7 cells that express low levels of CA1.

FIG. 4d is a diagram of analysis of effects of MKN7 and MKN7 with forcefully expressed CA1, on induction of anoikis by culturing them under monolayer or suspension conditions.

FIG. 4e is a diagram of analysis of effects of MKN7 and MKN7 with CA1-encapsulating exosomes added to a culture medium, on induction of anoikis under monolayer or suspension conditions.

### DESCRIPTION OF EMBODIMENTS

[Search for Novel Marker]

The search method for a novel marker will be described. Venous blood was collected from 48 patients with gastric cancer and 10 healthy subjects according to a conventional method and centrifuged at 4° C., 3,000 g for 5 minutes to obtain serum. The serum was stored at −80° C. until the time of use. Each 100 μl of serum was purified using size-exclusion chromatography, EVSecond columns (GL Sciences Inc.).

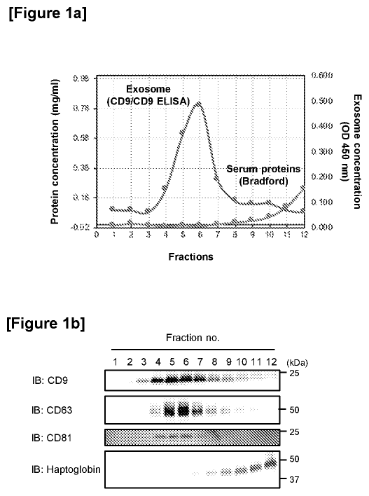

Fractions eluted from size-exclusion chromatography were collected in an amount of 100 μl each, and amounts of exosomes and serum proteins in each fraction were quantified (FIG. 1a). The exosomes were detected by CD9/CD9 sandwich ELISA, and the serum proteins were determined by protein quantification based on the Bradford method. The results indicated that fractions 4 to 7 were enriched in exosomes despite their low total protein content.

Besides, exosome markers CD9, CD63, and CD81, and a representative serum protein marker haptoglobin were analyzed by Western blotting. While these exosome markers are detected in the fractions 4 to 7, haptoglobin is detected in fractions 8 or later. Therefore, it was shown that the exosomes were separated from the serum proteins and purified with EVSecond columns. Note that the antibodies used are as follows: anti-CD9 antibody: monoclonal antibody (12A12, Shionogi & Co., Ltd.); anti-CD63 antibody: monoclonal antibody (8A12, Shionogi & Co., Ltd.); anti-CD81 antibody: monoclonal antibody (12C4, Shionogi & Co., Ltd.); and anti-haptoglobin antibody: polyclonal antibody (A0030, DAKO).

The purified exosomes were used to search a novel marker by mass spectrometry. The exosomes were dissolved in a denaturing solution (HEPES-NaOH, pH 8.0, 12 mM sodium deoxycholate, and 12 mM sodium N-lauroylsarcosinate), DTT was added thereto so as to reach 20 mM, the mixture was heated at 100° C. for 10 minutes, then iodoacetamide was added thereto so as to reach 50 mM, and alkylation was performed at room temperature for 45 minutes. Proteins derived from the thus obtained exosomes were digested with immobilized trypsin (Thermo Scientific) at 37° C. overnight with shaking. After removal of sodium deoxycholate and sodium N-lauroylsarcosinate with ethyl acetate, the obtained peptides were desalted by Oasis HLB μ-elution plate (Waters) to perform mass spectrometry.

The mass spectrometry was performed with an LTQ-Orbitrap-Veros Mass Spectrometer (Thermo Scientific) connected to UltiMate 3000 RLSC nano-flow HPLC (Thermo Scientific) equipped with 0.075×150 mm C18 tip-column (Nikkyo Technos). Analytical conditions are as follows.

Peptides were separated using a two-step gradient consisting of 2 to 35% and 35 to 95% acetonitrile concentrations with 0.1% formic acid at 250 nl/min for 95 minutes and 15 minutes, respectively. HPLC eluates were ionized with a spray voltage of 2 kV, and spectra in the 350 to 1,500 m/z range were analyzed in full MS ion scan mode with a resolution of 60,000. CID MS/MS scans were obtained in Data dependent acquisition (DDA) mode with the Dynamic exclusion function enabled.

Protein identification and quantitation were performed using Proteome Discoverer 2.2 software (Thermo Scinentific). MS/MS data were analyzed by SEQUEST (Thermo Scinentific) search engine, and a false discovery rate was set to less than 1% as a peptide identification threshold. For protein quantification and data standardization, default parameters of the Proteome Discoverer 2.2 software were used, and a Minora Feature Detector node and a Feature Mapper node after a Precursor Ions Quantifier node were used in processing workflow and consensus workflow, respectively.

Although an example of the search for a novel marker for gastric cancer is shown here, the methods shown in the example make it possible to easily purify and analyze exosomes from a small amount of blood sample. Therefore, the same method can be used to search markers for any disease, not just gastric cancer. This can be a useful method for searching novel markers in blood because biomarkers in blood samples can be searched for and used to examine for even diseases whose tissue samples are not easily obtained.

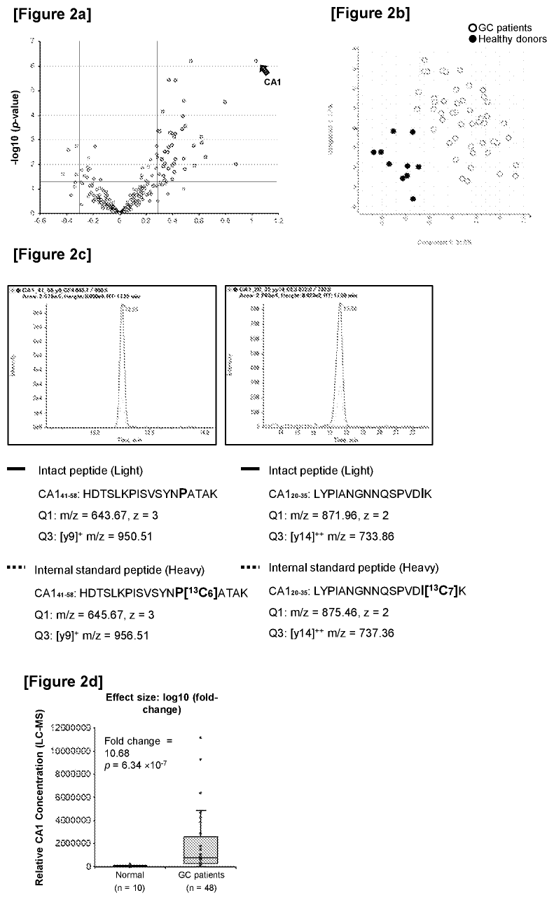

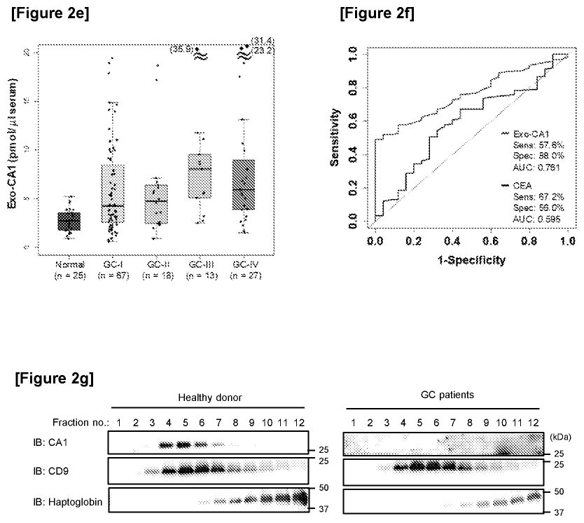

As a result of mass spectrometry on serum exosomes derived from 48 patients with gastric cancer and 10 healthy subjects, 1,281 proteins were identified, of which 816 proteins were extracted as intra-exosomal proteins. FIG. 2a shows a volcano plot comparing the exosomal proteins detected from exosomes in the serum of patients with gastric cancer and healthy subjects (p<0.05, effect size >2.0, significant value >50%). Of the 816 exosomal proteins, 40 proteins exhibited significantly enhanced expression in the exosome samples obtained from patients with gastric cancer, and 4 proteins exhibited decreased expression (Table 1). The 44 proteins with significant differences between patients with gastric cancer and healthy subjects were analyzed by partial least squares regression (FIG. 2b). The results revealed that these proteins could be clearly distinguished between patients with gastric cancer and healthy subjects.

TABLE 1

| | | | | |
|---|---|---|---|---|
| UniProt ID | Protein names | Gene names | Effect Size | p-Value |

40 up-regulated exosomal proteins in GC patients' sera

| | | | | |
|---|---|---|---|---|
| P00915 | Carbonic anhydrase 1 | CA1 | 10.6836 | 6.34E−07 |
| Q8NGR3 | Olfactory receptor 1K1 | OR1K1 | 7.5248 | 9.68E−03 |
| P02042 | Hemoglobin subunit delta | HBD | 6.2537 | 2.96E−05 |
| P00918 | Carbonic anhydrase 2 | CA2 | 4.4581 | 5.13E−03 |
| P30041 | Peroxiredoxin-6 | PRDX6 | 4.1673 | 1.34E−03 |
| P08519 | Apolipoprotein(a) | LPA | 4.1396 | 7.65E−04 |
| P01130 | Low-density lipoprotein receptor | LDLR | 3.6655 | 1.12E−02 |
| P32119 | Peroxiredoxin-2 | PRDX2 | 3.6256 | 1.71E−03 |
| O14672 | Disintegrin and metalloproteinase domain-containing | ADAM10 | 3.4512 | 6.15E−07 |
| | protein 10 | | | |
| Q96DT5 | Dynein heavy chain 11 axonemal | DNAH11 | 3.1161 | 2.78E−04 |
| P05186 | Alkaline phosphatase tissue-nonspecific isozyme | ALPL | 3.1001 | 1.58E−02 |
| P50895 | Basal cell adhesion molecule | BCAM | 3.0428 | 5.90E−03 |
| Q6UVY6 | DBH-like monooxygenase protein 1 | MOXD1 | 3.0402 | 2.62E−05 |
| Q6WKZ4 | Rab11 family-interacting protein 1 | RAB11FIP1 | 3.0043 | 1.07E−04 |
| P30626 | Sorcin | SRI | 2.9773 | 3.62E−04 |
| Q8IWS0 | PHD finger protein 6 | PHF6 | 2.6437 | 3.73E−06 |
| P11142 | Heat shock cognate 71 kDa protein | HSPA8 | 2.6365 | 6.18E−03 |
| P23229 | Integrin alpha-6 | ITGA6 | 2.6288 | 5.71E−04 |
| P00441 | Superoxide dismutase [Cu—Zn] | SOD1 | 2.5892 | 2.06E−02 |
| Q6SZW1 | Sterile alpha and TIR motif-containing protein 1 | SARM1 | 2.5836 | 7.54E−04 |
| P30456 | HLA class I histocompatibility antigen A-43 alpha chain | HLA-A | 2.5743 | 9.50E−03 |
| Q9BVS4 | Serine/threonine-protein kinase RIO2 | RIOK2 | 2.5164 | 5.63E−04 |
| P17661 | Desmin | DES | 2.5015 | 4.10E−02 |
| P18462 | HLA class I histocompatibility antigen A-25 alpha chain | HLA-A | 2.5012 | 1.11E−02 |
| P30450 | HLA class I histocompatibility antigen A-26 alpha chain | HLA-A | 2.5012 | 1.11E−02 |
| P30457 | HLA class I histocompatibility antigen A-66 alpha chain | HLA-A | 2.5012 | 1.11E−02 |
| P35443 | Thrombospondin-4 | THBS4 | 2.4840 | 1.70E−03 |
| Q9NZR2 | Low-density lipoprotein receptor-related protein 1B | LRP1B | 2.4028 | 5.25E−04 |
| P04040 | Catalase | CAT | 2.3546 | 3.60E−06 |
| Q96J66 | ATP-binding cassette sub-family C member 11 | ABCC11 | 2.3441 | 2.02E−03 |
| P54652 | Heat shock-related 70 kDa protein 2 | HSPA2 | 2.3333 | 1.48E−02 |
| P60953 | Cell division control protein 42 homolog | CDC42 | 2.3149 | 3.26E−03 |
| P62834 | Ras-related protein Rap-1A | RAP1A | 2.2005 | 9.50E−03 |
| P35606 | Coatomer subunit beta′ | COPB2 | 2.1931 | 3.04E−03 |
| P27701 | CD82 antigen | CD82 | 2.1768 | 4.53E−02 |
| Q16635 | Tafazzin | TAZ | 2.1413 | 1.23E−02 |
| P15144 | Aminopeptidase N | ANPEP | 2.1357 | 2.94E−02 |
| Q07954 | Prolow-density lipoprotein receptor-related protein 1 | LRP1 | 2.1132 | 7.08E−05 |
| A6NIZ1 | Ras-related protein Rap-1b-like protein | | 2.0843 | 6.30E−03 |
| P35613 | Basigin | BSG | 2.0113 | 4.49E−04 |

4 down-regulated exosomal proteins in GC patients' sera

| | | | | |
|---|---|---|---|---|
| Q4KWH8 | 1-phosphatidylinositol 4 5-bisphosphate | PLCH1 | 0.4723 | 2.63E−02 |
| | phosphodiesterase eta-1 | | | |
| A6NNZ2 | Tubulin beta-8 chain-like protein LOC260334 | | 0.4704 | 1.82E−03 |
| Q6YN16 | Hydroxysteroid dehydrogenase-like protein 2 | HSDL2 | 0.4086 | 2.54E−03 |
| O43790 | Keratin type II cuticular Hb6 | KRT86 | 0.3724 | 2.46E−02 |

t-test: p < 0.05, N < C: 2-fold, and valid value > 50%

Table 1 shows the proteins with significant differences between patients with gastric cancer and healthy subjects. There were 40 proteins with enhanced expression and 4 proteins with decreased expression in patients with gastric cancer. Therefore, any of the exosomal proteins can be analyzed to screen patients with gastric cancer.

Of these 44 proteins, carbonic anhydrase-1 (hereinafter described as CA1) was a biomarker with the biggest difference in exosomes obtained from groups of patients with gastric cancer and healthy subjects (FIG. 2a, Table 1). The CA1 levels contained in the exosomes were significantly different between patients with gastric cancer and healthy subjects, which were p=6.34×10⁻⁷ and fold change=10.68 (FIG. 2d). Therefore, a study of the usefulness of CA1, a marker for detecting gastric cancer, was conducted.

[Usefulness of CA1, Novel Gastric Cancer Biomarker]

In order to perform a quantitative analysis of CA1, analysis was performed by multiple reaction monitoring (MRM). Absolute quantification of the CA1 levels in the serum exosomes of 25 healthy subjects and patients with gastric cancer at stage classification I to IV (stage I: 67, II: 18, III: 13, and IV: 27) was performed (FIGS. 2c and 2e). The exosomal CA1 levels showed significantly higher values compared to the healthy subject group even in stage I, which is an early stage of gastric cancer, and showed further higher values as the disease progressed to more advanced stages. Therefore, by quantifying exosomal CA1 in the blood samples, gastric cancer can be tested.

Next, the sensitivity and specificity of gastric cancer detection by CA1 were analyzed through ROC curves (FIG. 2f). The sensitivity and specificity of gastric cancer detection by exosomal CA1 were 57.6% and 88.0%, respectively, and the area under curve (AUC) was 0.761. The AUC of CEA, an existing marker, was 0.595, indicating that exosomal CA1 is a marker with a superior ability to detect gastric cancer compared to CEA, the existing marker.

In order to confirm that exosomal CA1 is specifically detectable in serum from patients with cancer, analysis was performed by Western blotting. Exosomes were purified using the EVSecond column and analyzed for the presence of CA1, the exosome marker CD9, and the serum protein marker haptoglobin in each fraction (FIG. 2g). Note that the serum samples used were a mixture of serum from 6 patients with cancer or serum from 14 healthy subjects. For the detection of CA1, an anti-CA1 monoclonal antibody (ab108367, Abcam) was used.

In the analysis by Western blotting, CA1 was detected in serum samples from patients with gastric cancer, but not in serum samples from healthy subjects. CA1 was also detected in fractions in which an exosome marker CD9 was detected, that is, in the exosome fraction, but not fractions in which a serum protein haptoglobin was detected. In other words, CA1 was shown to be a specific marker as a gastric cancer marker contained in exosomes. The fact that it was detected using an antibody in the purified exosome fraction suggests that it can also be detected by a conventional method used in a clinical setting, such as ELISA. In addition, although serum was used here, it is obvious that plasma can also be used.

[Detection of CA1 in Gastric Cancer Tissues]

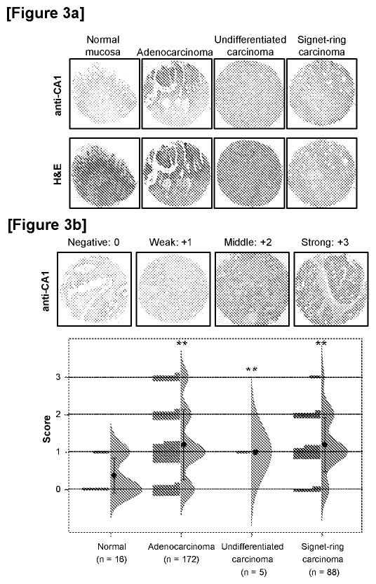

If CA1 expression can be specifically detected in gastric cancer tissues, it would be even more useful as a biomarker. Therefore, it was examined whether the CA1 expression could be detected in the gastric cancer tissues (FIG. 3).

Histological staining was performed with a CA1 antibody on 304 samples using a gastric cancer tissue microarray (US Biomax) to examine CA1 expression. Histological classification of the samples is as follows: adenocarcinoma: 172 cases; undifferentiated carcinoma: 5 cases; signet ring cell carcinoma: 80 cases; mucinous adenocarcinoma: 12 cases; malignant stromal tumor: 9 cases; carcinoid: 3 cases; and squamous cell carcinoma: 1 case.

In addition, 16 cases of normal gastric tissue were used as controls. Sections were deparaffinized, an anti-CA1 antibody (LifeSpan BioSience, Inc.) was used as a primary antibody, and EnVision™+ System (DAKO) was used for detection.

Staining could be performed in 281 cases, excluding those in which staining could not be performed. Expression of CA1 was found in 130 of 172 cases (75.6%) of gastric adenocarcinoma, 5 of 5 cases (100.0%) of undifferentiated carcinoma, 72 of 85 cases (84.7%) of signet ring cell carcinoma. In contrast, the expression of CA1 was either completely undetectable or only detected at low levels in normal mucosa (FIG. 3a).

Besides, staining intensity was classified into four levels from 0 to 3 in histological staining, and examinations of the staining intensity were conducted in adenocarcinoma, undifferentiated carcinoma, signet ring cell carcinoma, and normal tissue (FIG. 3b). Compared to the normal mucosa, the staining intensity of CA1 was shown to be significantly higher in all adenocarcinoma, undifferentiated carcinoma, and signet ring cell carcinoma. The staining of CA1 in gastric cancer tissues suggests that CA1, which is encapsulated in exosomes circulating in the blood, is secreted from the gastric cancer tissues. The fact that CA1 expression is also found in the gastric cancer tissues in histological staining indicates that CA1 can be used as a marker in pathological diagnosis as well.

[Examination Using Cell Lines]

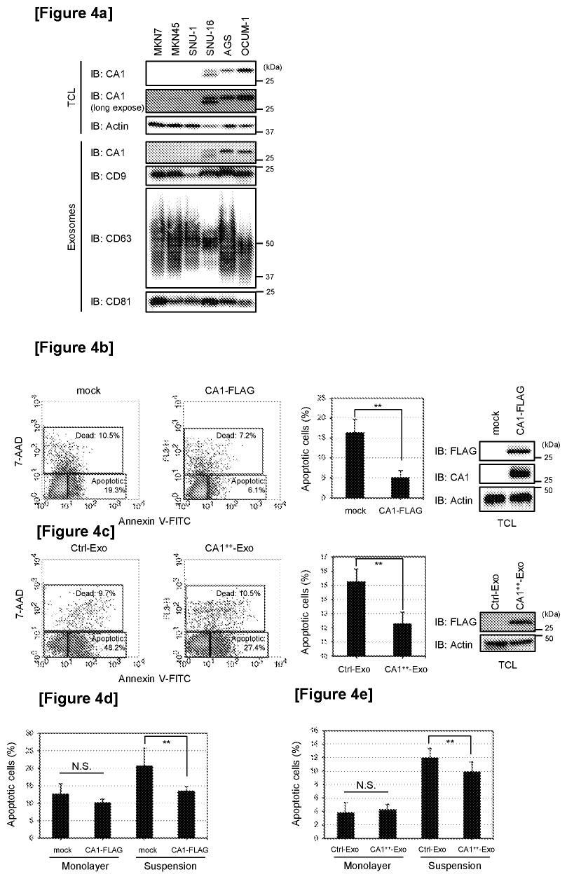

Expression of CA1 was examined using human gastric cancer cell lines. The CA1 expression of the human gastric cancer cell lines was analyzed by Western blotting using total cell lysate (TCL) (FIG. 4a, TCL). Histological types of the gastric cancer cell lines used were six: differentiated adenocarcinomas (MKN7 and AGS), poorly differentiated adenocarcinoma (MKN45), metastatic gastric cancers (SNU-1 and SNU-16), and scirrhous gastric cancer (OCUM-1). In SNU-16, OCUM-1, and AGS, CA1 was detected at a position of 29 kDa. Besides, low levels of CA1 expression were observed in MKN7 and MKN45 while no expression was observed in SNU-1.

Furthermore, exosomes were obtained from the culture supernatant of the gastric cancer cell line by ultracentrifugation and analyzed for CA1 expression by Western blotting (FIG. 4a, Exosomes). Exosomes obtained from cell lines endogenously expressing CA1 were found to contain CA1. This result indicates that CA1-encapsulating exosomes are secreted from cells expressing CA1. Note that CD9, CD63, and CD81 are exosome markers.

[Functional Analysis of CA1]

Using SNU-1 cells that did not express CA1, 3′-FLAG-tagged CA1, CA1 with a FLAG tag fused to its 3′ end was expressed therein for analysis. Staurosporine (STS), a kinase inhibitor, was added to the above cells at 1.0 μM to induce apoptosis (FIG. 4b). Apoptosis was detected with Annexin V, 7AAD kit (BD Bioscience), and analyzed by flow cytometry, BD FACSCalibur (BD Bioscience).

In 19.3% of SNU-1 cells that do not express CA1, apoptosis is induced within 3 hours after the start of staurosporine treatment. However, in cells with forcedly expressed CA1, cells in which apoptosis was induced were significantly decreased to 6.1%. This indicates that resistance to apoptosis is acquired by expressing CA1.

Exosomes were isolated from SNU-1 cells with 3′-FLAG-tagged CA1 forcefully expressed and added to an MKN7 culture medium, then apoptosis was induced by staurosporine in the same manner as above to analyze the effects of the addition of exosomes containing CA1 (FIG. 4c). The results revealed that the percentage of cells in which apoptosis was induced significantly decreased in the cells to which exosomes were added. Therefore, it is revealed that apoptosis resistance is also acquired even with exosomes encapsulating CA1.

Next, the effects of CA1 on anoikis were analyzed. Anoikis refers to apoptosis that is derived from anchorage dependence, which is caused by the inability to adhere to an extracellular matrix or by inappropriate adhesion thereto. In tumors, anoikis resistance is considered to be a property closely related to the invasion and metastasis of cancer cells.

MKN7 cells or MKN7 cells with CA1 forcefully expressed by 3′-FLAG-tagged CA1 were cultured under monolayer culture or suspension culture conditions to analyze the percentage of cells in which anoikis was induced by Annexin V, 7AAD staining (FIG. 4d). In the suspension culture, CA1 expression resulted in a significant decrease in the percentage of cells in which anoikis was induced.

Then, CA1-encapsulating exosomes were added to the culture supernatant of MKN7 cells and cultured in the same manner in monolayer culture or under suspension culture conditions to analyze the proportion of cells in which anoikis was induced (FIG. 4e). The addition of exosomes containing CA1 to the culture medium showed a significant decrease in the percentage of the cells in which anoikis was induced in the suspension culture. These results indicate that CA1 is also involved in resistance to anoikis.

As indicated above, the novel gastric cancer marker CA1 can detect gastric cancer with high sensitivity and specificity. It is also a marker closely related to apoptosis and anoikis resistance, which are associated with metastasis. Since the examination can be performed using blood samples, CA1 is a particularly useful marker for examining for recurrence and metastasis of gastric cancer.

Here, detailed analysis of CA1 was performed, including its function, and any of the proteins listed in Table 1 with significant differences in expression between patients with gastric cancer and healthy subjects can be used to detect gastric cancer. Particularly, 40 proteins exhibiting enhanced expression in patients with gastric cancer can be suitable markers for detecting gastric cancer. If a plurality of the markers listed in Table 1 are used for detection, the detection of gastric cancer can be performed with higher accuracy. As shown in the examples, it is possible to perform the detection of gastric cancer with high sensitivity in a minimally invasive method using blood.

1-15. (canceled)

16. An examination method for gastric cancer, comprising examining for expression of carbonic anhydrase-1 (CA1).

17. The examination method for gastric cancer according to claim 16, wherein the protein expression is to detect an amount of protein encapsulated in an exosome in a blood sample.

18. The examination method for gastric cancer according to claim 17, wherein the blood sample is serum or plasma.

19. The examination method for gastric cancer according to claim 16, wherein detection of the protein expression is performed by mass spectrometry.

20. The examination method for gastric cancer according to claim 16, wherein detection of the protein expression is performed using an antibody.

21. The examination method for gastric cancer according to claim 16, wherein detection of the protein expression is performed by tissue staining.

22. A search method for a disease marker, comprising: isolating exosomes from blood samples of patients suffering from a certain disease and of healthy subjects each by size-exclusion chromatography and identifying proteins with differences in expression between the patients and the healthy subjects by mass spectrometry to search a novel diseases marker.

23. A method for diagnosing gastric cancer, comprising detecting gastric cancer by collecting blood from a subject, detecting and quantifying an amount of CA1.

24. The method for diagnosing gastric cancer according to claim 23, wherein the detection of CA1 is by mass spectrometry or immunological detection method using an antibody.
